(19) United States
(12) Patent Application Publication
(10) Pub. No.: US 2019/0070019 A1
(43) Pub. Date: Mar. 7, 2019

(54) ORTHOPEDIC ANGULAR MEASURING INSTRUMENT

(71) Applicant: Smith & Nephew, Inc., Memphis, TN (US)

(72) Inventors: Phillip Frederick, Germantown, TN (US); Rachel Goss, Lakeland, TN (US); John Clausen, Germantown, TN (US); Russell Walter, Memphis, TN (US); Kevin Belew, Hernando, MS (US)

(21) Appl. No.: 16/079,368

(22) PCT Filed: Feb. 24, 2017
(86) PCT No.: PCT/US2017/019424

§ 371 Date: Aug. 23, 2018

Related U.S. Application Data
(60) Provisional application No. 62/299,267, filed on Feb. 24, 2016.

(51) Int. Cl. A61F 2/46; A61B 90/00

(52) U.S. Cl. CPC A61F 2/4657; A61F 2/4609; A61F 2002/4668; A61B 2090/3966; A61B 2090/3983; A61B 90/39

(57) ABSTRACT

An orthopedic angular measuring device including an elongated shaft having a longitudinal axis and configured for attachment to a bone engaging member, at least one marker associated with the shaft which is intraoperatively visible to determine the position or orientation of the bone engaging member relative to an image as the device is displaced relative to an angle or orientation. In one embodiment, the angular measure device is configured to attach to an acetabular cup for insertion of the cup into a patient's hip or acetabulum.

### CROSS-REFERENCE TO RELATED APPLICATIONS

This application claims the benefit of U.S. Provisional Application No. 62/299,267 filed Feb. 24, 2016, the contents of which are incorporated herein by reference in their entirety.

### FIELD OF THE INVENTION

The present invention relates to instrumentation and methods for use in orthopedic surgical procedures, and more particularly, but not exclusively, relates to instrumentation and methods for use in identifying the angular position/orientation of an acetabular implant during hip surgery.

### BACKGROUND

While regions of the human anatomy are intended to naturally articulate relative to one another in a smooth and non-abrasive manner, over time, the ease by which these anatomic regions are able to articulate degenerates in quality. Whether such problems arise from an injury, stress or a degenerative health problem, the natural articulation of these anatomical regions is often times no longer possible for these affected individuals. To correct these defects and restore normal articular movement of these anatomical regions, it may be desirable to replace the affected regions with a prosthetic component. For instance, it may become necessary to replace a patient's acetabulum with a prosthetic component if its articulation with the proximal femur becomes rough, abrasive or damaged. To accurately install an acetabular cup in the acetabulum in accordance with a pre-operatively defined orientation, it is necessary to monitor the angular position or orientation of the acetabular cup relative to the patient's anatomy as part of the hip surgery. However, in practice, there is a high level of variability in terms of accurately placing the cup in line with its targeted pre-operatively planned orientation. For example, if the patient's body moves during the surgical procedure, it may be difficult to quantify the angular rotation and how it would affect the planned positioning of the acetabular implants.

Conventional angular positioning devices are typically used for direct visualization purposes and have been utilized in different phases of treatment, including pre-operative treatment, intra-operative treatment, and post-operative treatment. Some of these direct visualization designs include levels and alignment tools which are positioned parallel with or perpendicular to planes or items such as the floor of the operating room, the alignment of the patient's spine, or the position/orientation of the operating room's surgical table. Still other designs utilize intra-operative imaging techniques to visualize the acetabular implant or the acetabular trial inside the patient's body for purposes of measuring associated angles, or utilize CT, MRI, or X-ray imaging techniques. These images can then be used to create a device which is a negative of the patient's acetabular or femoral anatomy. Generally, these devices are patient specific designs that are meant to be used with direct visualization techniques As a result, they most be inserted and physically attached to the patient'bony anatomy before any implant can be positioned. However, these processes are not very feasible for minimally invasive techniques.

Despite some advancement in the surgical field, there still remains a need to provide an improved instrument and method for use in identifying the angular position/orientation of an acetabular implant during hip surgery. The present invention satisfies this need and provides other benefits and advantages in a novel and unobvious manner.

### SUMMARY

While the actual nature of the invention covered herein can only be determined with reference to the claims appended hereto, certain forms of the invention that are characteristic of the embodiments disclosed herein are described briefly as follows.

It is one object of the present invention to provide an improved instrument and method for use in identifying the angular position/orientation of an acetabular implant during hip surgery. Further embodiments, forms, features, aspects, benefits, objects, and advantages of the present invention will become apparent from the detailed description and figures provided herewith.

In accordance with one form of the present invention, an orthopedic angular measuring device is provided including an elongated shaft having a longitudinal axis and configured to attach to an acetabular cup, and at least one marker associated with the shaft that is intra-operatively visible to determine the position and/or orientation of the acetabular cup relative to a preoperative image as the device is moved relative to the longitudinal axis.

In accordance with another form of the present invention, a method of measuring the angular position of an acetabular cup is provided, including providing an insertion device configured to attach to the acetabular cup, placing a marker on the insertion device that is intra-operatively visible by an imaging device, rotating the insertion device from a first position to a second position, and determining the angular positon of the acetabular cup relative to a preoperative image of a patient's anatomy after the device is rotated to the second position.

In accordance with a further form of the present invention, an orthopedic angular measuring device is provided for use with an imaging device. The measuring device includes an elongated shaft having a longitudinal axis and configured for attachment to a bone engaging member. At least one marker is associated with the shaft and which is intra-operatively visible with the imaging device to determine an orientation of the longitudinal axis relative to a reference angle as the elongated shaft moved relative to the reference angle.

In accordance with still another form of the present invention, an orthopedic insertion tool is provided including an elongated shaft having a longitudinal and configured for attachment to an acetabular cup. At least one marker is associated with the shaft and which is intraoperatively visible to determine the position and/or orientation of the acetabular cup relative to a preoperative image as the device is moved relative to the longitudinal axis.

In accordance with yet another form of the present invention, a method of measuring the angular position of a bone engaging member is provided, including providing an insertion device configured for attachment to the bone engaging member, placing a marker on the insertion device which is intraoperatively visible by an imaging device, moving the insertion device from a first orientation to a second orientation, and determining an angular orientation of the bone engaging member relative to a preoperative image of a patient's anatomy after the device is moved to the second orientation.

### BRIEF DESCRIPTION OF THE DRAWINGS

FIG. 1A is an elevational view of an orthopedic angular measuring device according to one form of the present invention positioned at a first angular orientation.

FIG. 1B is an elevational view of the orthopedic angular measuring device positioned at a second angular orientation.

FIG. 1C is an elevational view of the orthopedic angular measuring device positioned at a third angular orientation.

FIG. 2A is a second elevational view of the orthopedic angular measuring device positioned at the first angular orientation shown in FIG. 1A.

FIG. 2B is a second elevational view of the orthopedic angular measuring device positioned at the second angular orientation shown in FIG. 1B.

FIG. 2C is a second elevational view of the orthopedic angular measuring device positioned at the third angular orientation shown in FIG. 1C.

FIG. 3 is an elevational view of the orthopedic angular measuring device used in association with an acetabular cup to position the acetabular cup relative to the acetabulum of a patient.

FIG. 4 is an elevational view of an orthopedic angular measuring device according to another form of the present invention including an outrigger mechanism.

FIG. 5 is a rotated elevational view of the orthopedic angular measuring device of FIG. 4 illustrating a plurality of indicia markings.

FIG. 6 is an enlarged view of a portion of the orthopedic angular measuring device of FIG. 5 illustrating the plurality of indicia markings.

FIG. 7 is an elevational view of an orthopedic angular measuring device according to another form of the present invention.

FIG. 8 is an elevational view of an orthopedic angular measuring device according to another form of the present invention.

FIG. 9 is an elevational view of an orthopedic angular measuring device according to another form of the present invention in a first angular orientation.

FIG. 10 is an elevational view of the orthopedic angular measuring device of FIG. 9 in a second angular orientation.

FIG. 11 is an elevational view of the orthopedic angular measuring device of FIG. 9 in a third angular orientation.

FIG. 12 is an elevational perspective view of an orthopedic angular measuring device according to another form of the present invention.

### DESCRIPTION OF THE ILLUSTRATED EMBODIMENTS

For the purpose of promoting an understanding of the principles of the present invention, reference will now be made to the embodiments illustrated in the drawings and specific language will be used to describe the same. It will nevertheless be understood that no limitation of the scope of the invention is hereby intended. Any alterations and further modifications in the described embodiments, and any further applications of the principles of the invention as described herein are contemplated as would normally occur to one skilled in the art to which the invention relates.

The following descriptions and illustrations of non-limning embodiments of the present invention are exemplary in nature, it being understood that the descriptions and illustrations related thereto are in no way intended to limit the inventions disclosed herein and/or their applications and uses.

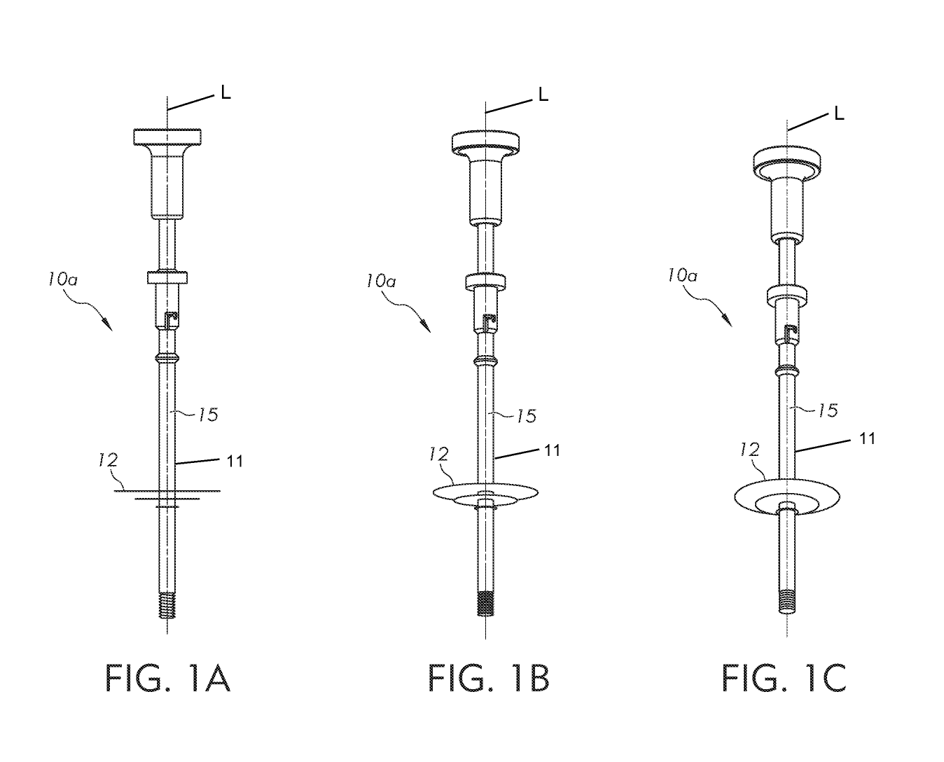

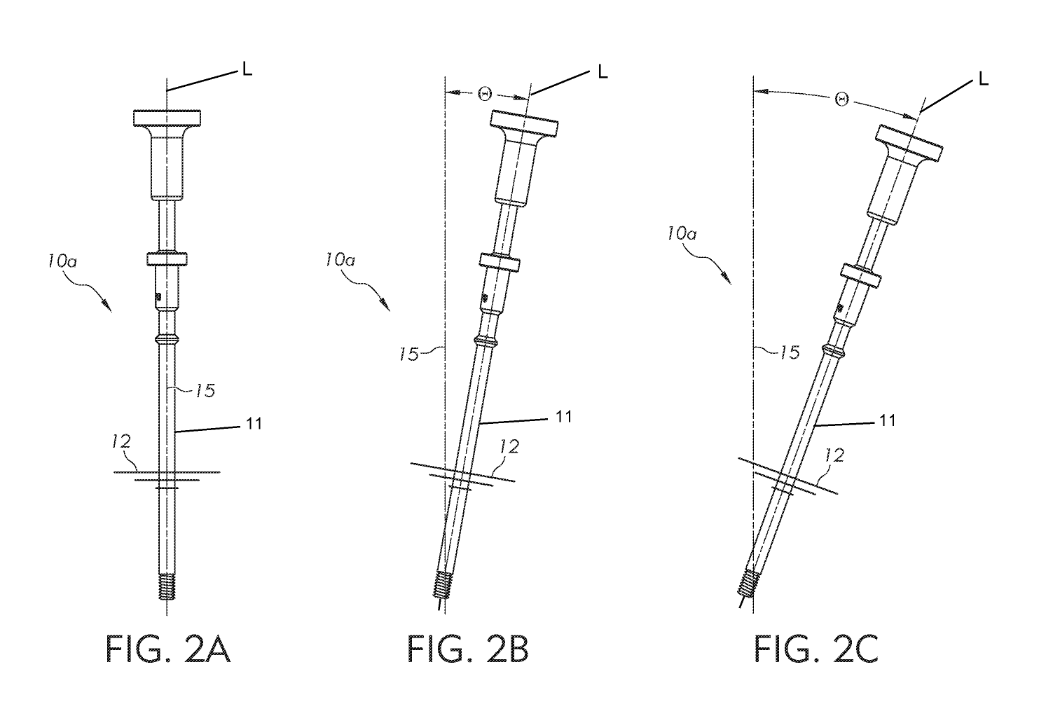

Referring to FIGS. 1-3, shown therein is an orthopedic angular measuring device 10a according to one form of the invention. The orthopedic angular measuring device 10a extends generally along a longitudinal axis L and includes an elongated handle or shaft 11 and one or more markers 12 attached to the elongated shaft 11. In addition to being used as to measure angular position/orientation, the angular measuring device 10a may also be used as an insertion and/or impaction device. Although a method for using the orthopedic angular measuring device 10a to identify the angular position/orientation of an attached implant, trial or other structures will be discussed below, it should be understood and appreciated that other methods and uses of the measuring device 10a are also contemplated as falling within the scope of the present invention.

In accordance with a first illustrative embodiment, the angular measuring device 10a controls how and where the implant, trial or other structures are positioned/oriented without the use of any patient specific or customized measurement components of instruments. Instead, the angular measuring device 10a, or an attachment to the device, creates a different visual outline, profile, pattern, indicia or shadow when rotated out of plane relative to an intraoperative image of the patient's anatomy. To accomplish this, radiopaque or radiolucent circles, disks, indicia or markers 12 may be used in association with the device 10a. As illustrated in FIGS. 1A-1C, the markers 12 are fixed to the device 10a and are each arranged along a plane substantially perpendicular to a longitudinal axis L of the device.

When the markers 12 are rotated out of plane, the circles/disks create observable oval shapes of different dimensions which intersect each other or other marks as identifiers. While it should be understood and appreciated that any geometric shapes, words, numbers, indicia and/or combinations thereof could be used in association with the markers 12, in accordance with certain aspects of the invention, circles are particularly useful because they do not require the instrument to be rotated in a specific manner or to a particular home position/orientation in order to show the image accurately. Circles (or disks) of different diameters, when observed in plane, appear as lines (see FIG 1A). The markers 12 are illustrated at different angular orientations in FIGS. 1A/2A, 1B/2B and 1C/2C, where FIGS. 1A/2A illustrates the device angled at 0° with respect to a reference axis 15. In FIGS. 1A/2A, the reference axis 15 is defined as a zero degree axis. When the markers 12 are observed by an imaging device at the 0° orientation illustrated in FIGS. 1A/2A, the observed markers 12 appear as parallel lines (see FIG. 1A).

FIGS. 1B/2B illustrate the device 10a rotated at an angle θ of 10° with respect to the reference axis 15 (i.e., a 10° angle defined between the longitudinal axis L and the reference axis 15), and FIGS. 1C/2C illustrate the device 10a rotated at an angle θ of 20° angle with respect to the reference axis 15 (i.e., a 10° angle defined between the longitudinal axis L and the reference axis 15). When rotated out of plane, the markers 12 become visible as ovals (see, for example, markers 12 of FIGS. 1B and 1C), where FIGS 1B/2B show the device 10a related at 10° with respect to the reference axis 15, and FIGS. 1C/2C show the device 10a rotated at 20° with respect to the reference axis 15. In other words, when the markers 12 are positioned at specific depths relative to one another, and when the device 10a is rotated out of plane, the markers 12 create the illusion of ovals which intersect when rotated out of plane at an angle θ. The intersections or tangential positions of the ovals are designed to occur at specific angular orientations of the device 10a, which in turn provide an indication of the angular orientation of the device 10a (i.e., at an angle θ). If the device 10a is provided with solid disks, the disappearance or overlap of smaller disks may also signify a particular angular orientation of the device 10a.

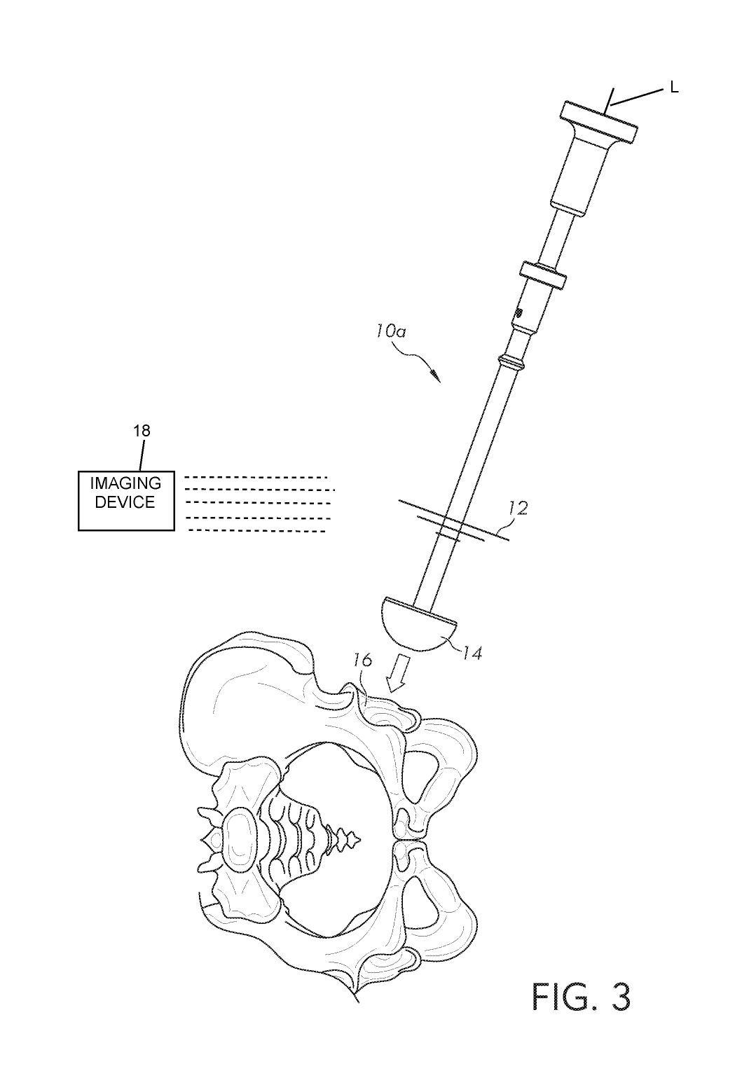

FIG. 3 illustrates a perspective view of the orthopedic angular measuring device 10a, as used in association with a bone engaging member 14 to position the bone engaging member 14 relative to a bone 16 of a patient. In the illustrated embodiment, the bone engaging member 14 is an acetabular cup configured to engage a socket of a patient's hip bone, and more particularly the patient's acetabulum. While an acetabular cup is illustrated for use in association with the device 10a, the present invention is not limited for use in association with an acetabular cup, but the use of other bone engaging members 14 in relation to other bones are also contemplated as falling within the scope of the present invention. An imaging device 18 is used in different embodiments to provide an indication of the angle θ of the device 10 with respect to the reference axis 15. Imaging devices 18 that may be used in association with the present invention to observe or visualize the markers 12 include, but are not limited to, x-ray imaging devices, ultrasound imaging devices, magnetic resonance imaging (MRI) devices, computed tomography (CT) imaging devices, fluoroscopic imaging devices, or other suitable imaging devices.

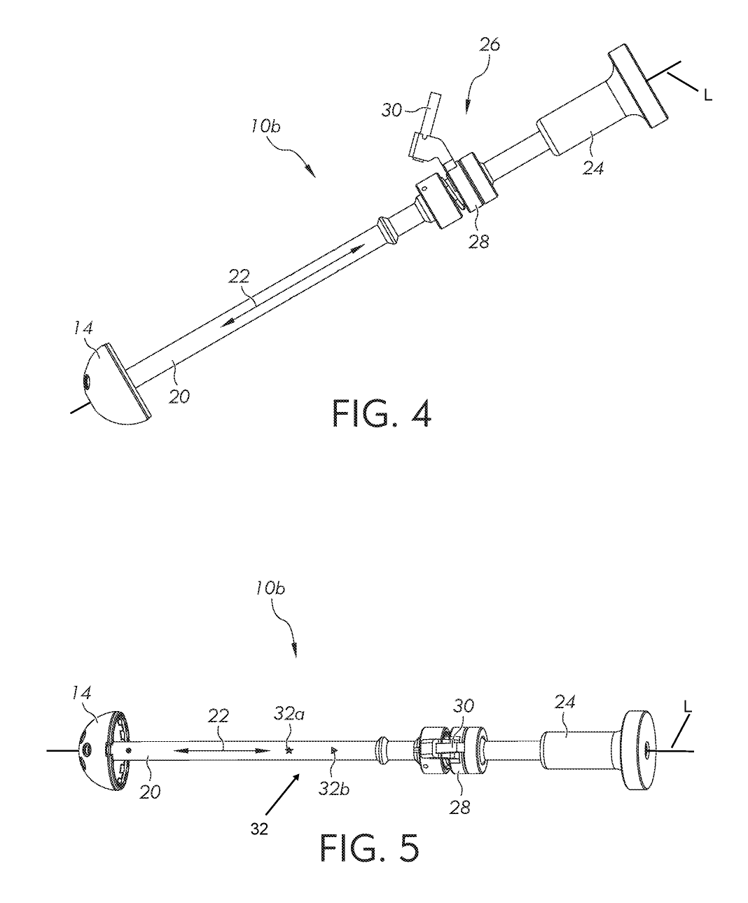

Referring to FIG. 4, shown therein is an orthopedic angular measuring device 10b according to another form of the invention. The orthopedic angular measuring device 10b extends generally along a longitudinal axis L and includes an elongated handle or shaft 20, and is illustrated at a zero angle with respect to a reference axis 22. The device 10b may include an impactor 24 attached to the proximal end of the shaft 20 which may be used to drive the bone engaging member 14 into bone. The shaft 20 supports an outrigger member 26 including a holder 28 having a cylindrical shape configured to surround a portion of the shaft 20. In one embodiment, the holder 28 is freely rotatable about and slidable along the shaft 20, and can be fixed at various locations along and about the shaft 20. In other embodiment, the interior of the holder 28 may support a bearing such that the holder 28 may freely rotate under the force of gravity.

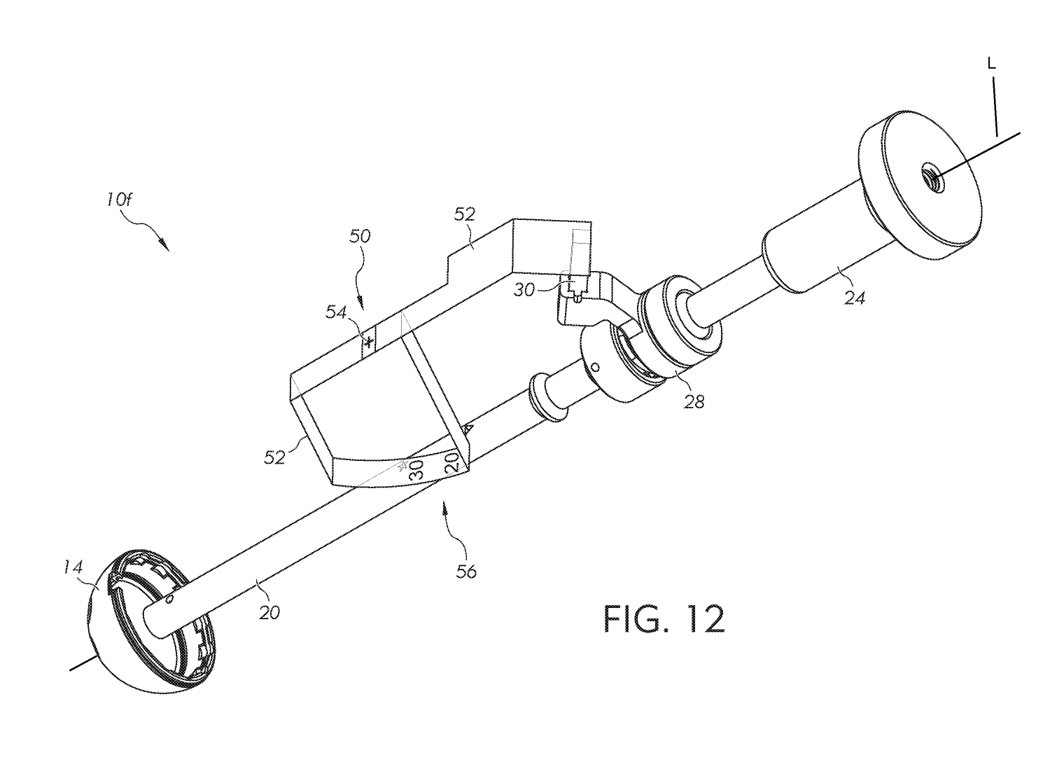

An alignment guide support 30 extends from and is fixedly coupled to the holder 28. The alignment guide support is either permanently fixed to the holder 28, or includes a coupler that enables the alignment guide support 30 to be releasably coupled to the holder 28 for attachment and removal of the holder 28 to/from the shaft 20. The support 30 is configured to provide a mount tor an alignment guide, as described below in association with FIG. 12.

FIG. 5 illustrates the orthopedic angular measuring device 10b of FIG. 4, but with the device rotated 90 degrees about the longitudinal axis L to illustrate a plurality of markings or visualization indicia 32. While a first marking 32a and a second marking 32b are illustrated, it should be understood that the inclusion of any number of masking or indicia are contemplated including one marking or three or more markings. Each of the markings 32a and 32b defines a channel or passage which extends entirely through the shaft 20 such that light passes through the channels. In other embodiments, the channel may extend only partway through the shaft 20. For each of the markings 32, the channel defines sidewalls which are configured to define a particular shape. For instance, marking 32a defines a star shape, and marking 32b defines a triangular shape.

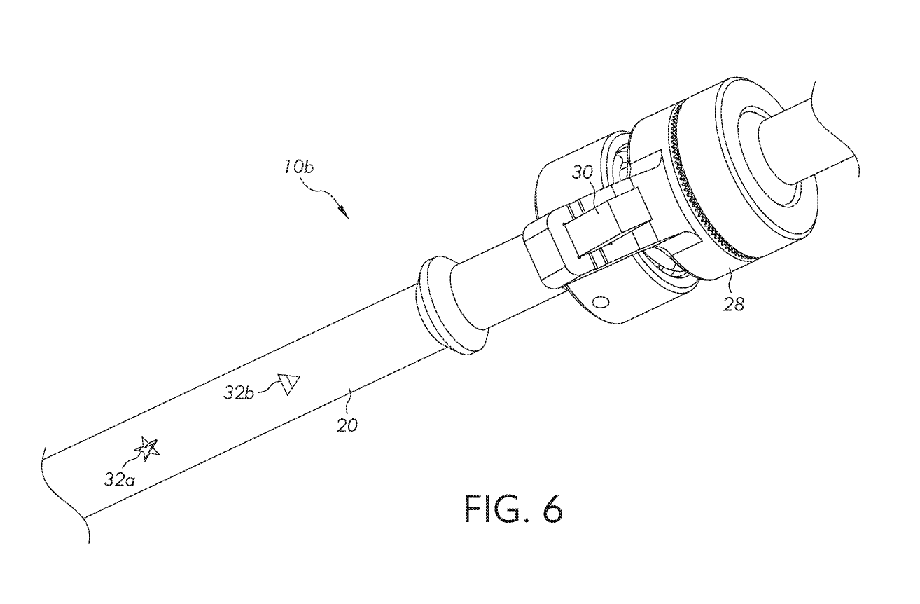

Each of the markings 32 formed in the shaft 20 is inclined relative to the longitudinal axis L. For example, in one embodiment, the marking 32a is inclined or oriented at 30 degrees relative to a line arranged perpendicular or normal to the longitudinal axis L, and the marking 32b is inclined or oriented at 20 degrees relative to a line arranged perpendicular or normal to the longitudinal axis L. The angled channels determine whether or not the shaft 20, and consequently the device 10b and the bone engaging member 14, are properly aligned at the correct/desired angular orientation relative to the reference axis 22. For example, to align the bone engaging member 14 at 30 degrees relative to the reference axis 22, the shaft 20 is angularly displaced until the interior sidewalks of the mark 32a are not visible (i.e., the channel defined by the mark 32a is in angular alignment with the imaging device). If, however, the shaft 20 is aligned at 20 degrees, then the interior sidewalls of the mark 32b are not visible (i.e., the channel defined by the mark 32b is in angular alignment with the imaging device). As can be seen in FIG. 6, the interior sidewalls of both the marks 32a and 32b are visible, and consequently the bone engaging member 14 is not aligned at either 20 degrees or at 30 degrees.

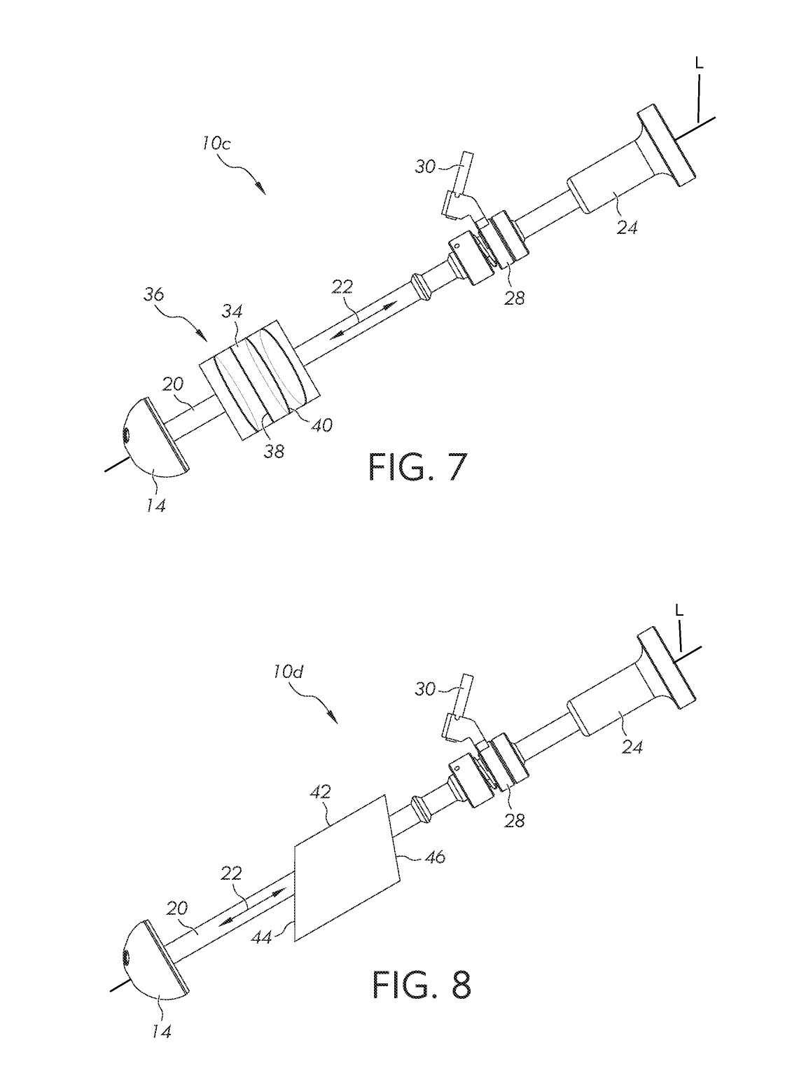

Referring to FIG. 7, shown therein is an orthopedic angular measuring device 10c according to another form of the invention. The orthopedic angular measuring device 10c includes a cylindrical marker 34 that is centered about the longitudinal axis L of the shaft 20. In one embodiment, the cylindrical marker 34 is formed of a radiolucent material having a plurality of radiopaque isoclines formed about the cylinder's diameter. Each of the isoclines is inclined with respect to the longitudinal axis L of the shaft 20 such that the appearance of the isoclines changes with respect to an observer upon a change in the angular orientation of the device 10c relative to a reference axis 22, which can be observed directly by an individual or via an imaging system. For example, an isocline 38 appears as a straight line to an observer when the shaft 20 is inclined at 30 degrees with respect to a reference axis or plane, and each of the remaining isoclines appears as a type of oval having a different appearance when the device 10c is inclined at 30 degrees. As the angle of the device 10c and the longitudinal axis L is changed, for instance to 15 degrees, an isocline 40 transitions from an oval shape to a straight line, thereby indicating that the shaft 20 is inclined at 15 degrees with respect to a reference axis or plane. With intra-operative imaging or direct viewing by an observer, the angular orientation of the device 10c may be determined from the shape of the radiopaque isoclines (i.e., when a particular isocline is observed as a straight line, which corresponds to a particular angular orientation of the device relative to the reference axis or plane).

Referring to FIG. 8, sown therein is an orthopedic angular measuring device 10d according to another form of the invention. The orthopedic angular measuring device 10d including a cylinder 42, with the shaft 20 extending through the cylinder 42, and where the cylinder 42 is fixed at an axial location along the shaft. The cylinder 42, in various embodiments, may be formed of a radiopaque or radiolucent material. In this embodiment, however, the ends of the cylinder 42 are inclined with respect to the longitudinal axis L of the shaft 20. The cylinder 42 includes a first end 44 defining a planar surface that is inclined at a first angle with respect to the longitudinal axis L. A second end 46 of the cylinder 42 defines a planar surface that is inclined at a second angle with respect to the longitudinal axis L. In this exemplary embodiment, the first end 44 is indicative of an angle of 30 degrees, and the second end 46 is indicative of an angle of 20 degrees. In one embodiment, the cylinder 42 is formed of a radiopaque material so that each of the ends 44 and 46 are viewable during an intraoperative procedure by an imaging device. In another embodiment, the cylinder 42 is formed of a radiolucent material and each of the ends 44 and 46 are coated or painted with a radiopaque material which is apparent or observable to an observer or by an imaging system.

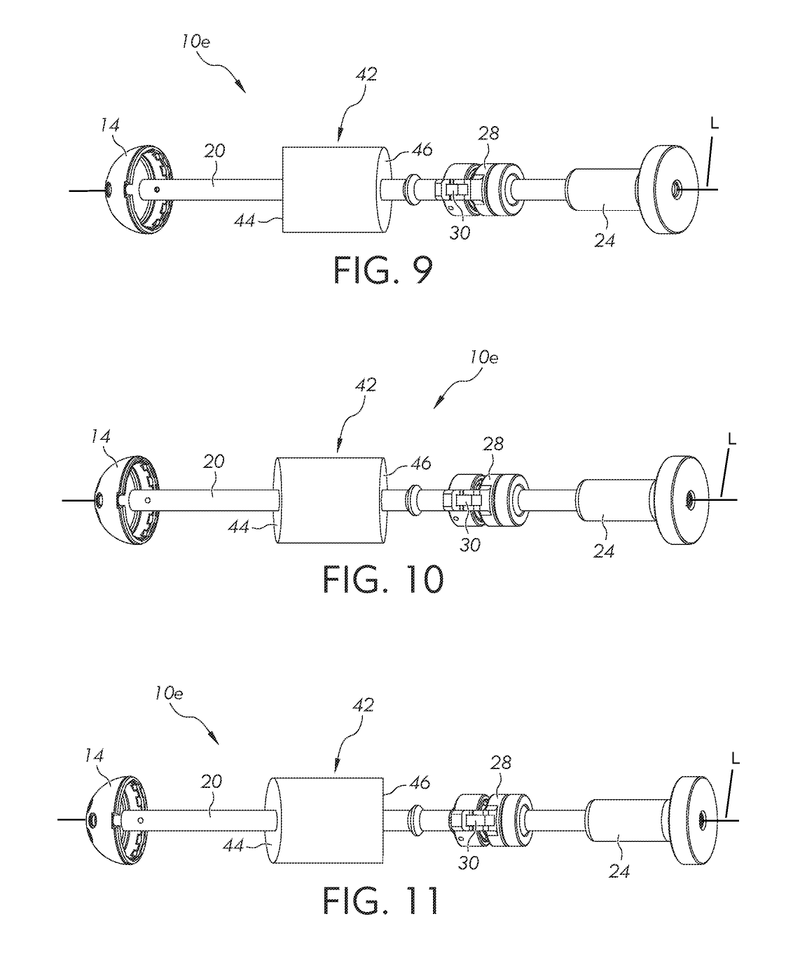

Referring to FIG. 9, shown therein is an orthopedic angular measuring device 10e according to another form of the invention. The orthopedic angular measuring device 10e illustrated in FIG. 9 is shown in a first angular orientation relative to a reference axis or plane. In this first angular orientation, the surface 44 appears as a straight line to indicate that the device 10e is aligned with the angle indicated by the surface 44. The surface 46, however, does not appear as a straight line, but instead provides at least a partial view of the surface 46. Since the surface 46 is viewable, the device 10e is not aligned with the angle indicated by the surface 46.

FIG. 10 illustrates an elevational view of the orthopedic angular measuring device 10e in a second angular orientation. This position is one in which the device 10e is not oriented at either of the predetermined angles indicated by the surface 44 and the surface 46. As can be seen, each of the surfaces of the surface 44 and the surface 46 are at least partially visible, which in turn indicates that the device 10e is located at a relatively undefined and approximate location. in this position, the device 10e is located between the angle indicated by the surface 44 and the angle indicated by surface 46.

FIG. 11 illustrates an elevational view of the orthopedic angular measuring device 10e in a third angular orientation. In this position, the surface 46 appears as a straight line to the observer or to the imaging device which indicates that the device 10e is aligned with the angle indicated by the surface 46, and with at least a portion of the surface 44 being visible, which in turn indicates that the device 10e is not at the angle indicated by surface 44.

Referring to FIG. 12, shown therein is an orthopedic angular measuring device 10f according to another form of the invention. The orthopedic angular measuring device 10f includes a support 28 that is configured to locate and support an alignment guide 50. The alignment guide 50, in one embodiment, is formed of a radiolucent material having a plurality of sides or edges including a radiopaque material. The alignment guide 50 is fixedly connected to the device 10f by the mount 30. In other embodiments, other supports or mounts may be used to fix the location of the guide 50 with respect to the shaft 20 so that a repeatable determination of the angle of the device 10f may be provided. In one embodiment, the alignment guide 50 is offset from the shaft 20 to enable the imaging device or an observer to determine the identity of any markings which appear on the shaft 20. In another embodiment, the alignment guide 50 is aligned with the shaft 20 to enable the imaging device or an observer to determine the identity of only marks provided by the alignment guide 50.

The alignment guide 50 includes an exterior surface 52 located about a perimeter of the guide 50. The exterior surface 52 is coated with a radiopaque material at certain portions of the perimeter to define a viewfinder 54, the location of which is defined by the absence of the radiopaque material. At a bottom portion 56 of the guide 50, one or more numbers or symbols that are indicative of angles of inclination are provided. In the illustrated embodiment, the numbers provided are 20 and 30, each of which corresponds to an angle of the device 10f with respect to the zero or reference axis. If the device 10f is angled at a 30 degree angle, the number 30 is seen through the viewfinder 54 by an observer or by an imaging device. If the device 10f is, however, angled at a 20 degree angle, the number 20 is seen through the viewfinder by an observer or by an imaging device.

In accordance with other embodiments of the present invention, instruments or trials having pre-determined shapes or markers visible by C-ARM or x-ray may be utilized. These devices may be matched to identify the patient specific bone engaging member placement, including acetabular placement, as determined either pre-operatively or intra-operatively, and may utilize a specialized radiopaque or radiolucent mark(s) or indicia. According to certain aspects of this process, the surgeon or other medical personnel would pre-operatively plan to place the bone engaging member, such as an acetabular implant, in a specific version and abduction angle. To accomplish this, an instrument may be utilized which, when positioned in the pre-determined implant orientation and viewed intra-operatively, would provide a visual shape that signifies the device is in the correct or incorrect position. In one or more embodiments, the markers are located more closely to the end of the tool to which the bone engaging member is located than to the impactor 24. However, other locations are also contemplated.

Moreover, intraoperative imaging can be utilized with overlays or templates to compare positions of bone engaging members, including acetabular components. This is an improvement to current patient specific guides which typically require direct mating with the anatomy, as well as additional preoperative and intraoperative surgical steps. In addition, surgeons who utilize intraoperative imaging can benefit from such a system as it does not require custom implants to be utilized and only requires viewing the intraoperative image to determine if the inserter, and thus the implant, is being positioned in the correct manner.

Various changes and modifications to the described embodiments described herein will be apparent to those skilled in the art, and such changes and modifications can be made without departing from the spirit and scope of the invention and without diminishing its intended advantages. Additionally, while the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

1. An orthopedic angular measuring device for use with an imaging device, comprising:
an elongated shaft having a longitudinal axis and configured for attachment to a bone engaging member, and
at least one marker associated with the elongated shaft, the marker being intra-operatively visible with the imaging device to determine an angular orientation of the longitudinal axis relative to a reference axis or plane as the elongated shaft is moved relative to the reference axis or plane.

2. The device of claim 1, wherein the at least one marker is fixedly coupled to the elongated shaft.

3. The device of claim 1, wherein the at least one marker is aligned with the longitudinal axis of the elongated shaft.

4. The device of claim 3, wherein the at least one marker comprises an aperture in the elongated shaft that is inclined at an angle of inclination with respect to the longitudinal axis.

5. The device of claim 1, further comprising an alignment guide extending from the elongated shaft; and
wherein the alignment guide is offset from the longitudinal axis of the elongated shaft.

6. The device of claim 1, wherein the at least one marker is either fixed at a predetermined angle relative to the longitudinal axis, or is incrementally adjustable at varying angles of inclination relative to the longitudinal axis.

7. The device of claim 1, wherein the at least one marker includes an alphanumeric character, a symbol, and/or a geometric pattern.

8. The device of claim 1, wherein the at least one marker is radiopaque or radiolucent.

9. The device of claim 1, wherein the at least one marker includes a plurality of circular elements, and wherein each circular element has a different diameter.

10. The device of claim 1, wherein the at least one marker comprises a solid disk.

11. The device of claim 1, wherein the at least one marker comprises a body having a first surface inclined at a first angle with respect to the longitudinal axis and a second surface inclined at a second angle with respect to the longitudinal axis, and wherein each of the first surface and second surface define a different angle of inclination relative to the longitudinal axis.

12. An orthopedic insertion instrument, comprising:
an elongated shaft having a longitudinal axis and configured for attachment to an acetabular cup; and
at least one marker associated with the shaft, the marker being intraoperatively visible to determine the position and/or angular orientation of the acetabular cup relative to a preoperative image as the device is moved relative to a reference axis or plane.

13. The instrument of claim 12, wherein the at least one marker is fixedly coupled to the elongated shaft.

14. The instrument of claim 12, wherein the at least one marker is radiopaque or radiolucent.

15. The instrument of claim 12, wherein the at least one marker includes a plurality of circular elements, and wherein each circular element has a different diameter.

16. The instrument of claim 12, wherein the at least one marker comprises a solid disk.

17. A method of measuring the angular position of a bone engaging member, comprising:
providing an insertion device configured for attachment to the bone engaging member;
placing a marker on the insertion device, the marker being intraoperatively visible by an imaging device;
displacing the insertion device from a first orientation to a second orientation; and
determining an angular orientation of the bone engaging member relative to a preoperative image of a patient's anatomy after the device is moved to the second orientation by observing a change in the marker.

18. The method of claim 17, wherein the placing a marker on the insertion device comprises placing at least one radiopaque or radiolucent marker on the insertion device.

19. The method of claim 17, wherein the displacing the insertion device comprises moving the device out of plane.

20. The method of claim 17, wherein the determining the angular position of the bone engaging member comprises observing a shape of the marker.
